(19) United States
(12) Patent Application Publication
(10) Pub. No.: US 2017/0258706 A1
(43) Pub. Date: Sep. 14, 2017

(54) LIP COMPOSITIONS CONTAINING FOOD POWDER

(71) Applicant: The Burt's Bees Products Company, Oakland, CA (US)

(72) Inventors: Cheryl Hili, Durham, NC (US); Amanda Biggam-Nobbe, Durham, NC (US); Abena Antwi, Durham, NC (US); Matthew T. Cooper, Durham, NC (US)

(21) Appl. No.: 15/456,145

(22) Filed: Mar. 10, 2017

Related U.S. Application Data
(60) Provisional application No. 62/308,113, filed on Mar. 14, 2016.

(51) Int. Cl. A61K 8/97; A61K 8/67; A61Q 1/06; A61K 8/92; A61Q 1/04

(52) U.S. Cl. CPC A61K 8/97; A61K 8/922; A61Q 1/04; A61Q 1/06; A61K 8/927; A61K 8/678; A61K 2800/522

(57) ABSTRACT

Disclosed herein are cosmetic lip compositions that contain dried food powders. The dried food powders are present in about 1% to about 20% and impart flavor to the lip compositions. The lip compositions can be solid (e.g., balms, lipsticks) or semi-solid (e.g, gels in tubes) and can be made using known manufacturing processes.

### CROSS-REFENCE TO RELATED APPLICATIONS

The application claims the benefit of U.S. Provisional Application No. 62/308,113, filed Mar. 14, 2016, which is hereby incorporated by reference in its entirety.

### BACKGROUND OF THE INVENTION

1. The Field of the Invention

The present invention is concerned with the utilization of dehydrated food powders as an ingredient in lip balms, lip sticks and other cosmetic preparations designed for lip application.

2. Description of Related Art

Flavors have been used as an ingredient in cosmetic lip preparations virtually since the first lip balm product was introduced. Numerous flavors and flavor combinations have been used in countless preparations; however flavor ingredients utilized have always been artificial or natural fragrances. These traditional flavor ingredients provide an odor (which the user interprets as flavor) but lack true taste. Additionally, sweeteners are often added to enhance the flavor experience of the lip preparation. Prior art examples cite other improvements to lip products but fail to solve the problem of real natural taste in a lip balm because they rely on fragrances and sweetener blends for taste.

### BRIEF SUMMARY OF THE INVENTION

Place holder

### DETAILED DESCRIPTION OF THE PREFERRED EMBODIMENTS

### I. DEFINITIONS

Before describing the present invention, it is to be understood that this invention is not limited to particularly exemplified compositions, systems or process parameters that may, of course, vary. It is also to be understood that particular embodiments of the invention may be described in detail and such detail is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

References herein to “one embodiment”, “one aspect” or “one version” of the invention include one or more such embodiment, aspect or version, unless the context clearly dictates otherwise.

As used in this specification and the appended claims, the singular forms “a”, “an”, and “the” include plural referents unless the context clearly dictates otherwise.

Unless defined otherwise, all technical and scientific terms used herein have the same meaning as commonly understood by one of ordinary skill in the art to which the invention pertains. Although it is possible that a number of methods and materials similar or equivalent to those described herein can be used in the practice of the present invention, the preferred materials and methods are described herein.

In the application, effective amounts are generally those amounts listed as the ranges or levels of ingredients in the descriptions, which follow hereto. Unless otherwise stated, amounts listed in percentage (“%'s”) are in weight percent (based on 100% active) of the active composition alone, unless otherwise indicated.

Disclosed herein are preferred ranges. It is to be understood that values representing upper or lower ceiling values (e.g., at least 10%) or values representing ranges (e.g., from 10% to 20%) are inclusive of all values therebetween, (e.g., at least 10% is inclusive of at least 8.5% and from 10% to 20% is inclusive of from 12.2% to 17%, etc.).

The term “naturally-derived” as used herein is meant to mean that the ingredient comes or is made from a renewable resource found in nature (i.e., Flora, Fauna, Mineral). Petroleum compounds are expressly excluded from the term “naturally-derived”.

The term “naturally-processed” as used herein means the ingredients are processed using only ecologically-friendly processes. Ecologically-friendly processing is minimal processing that maximizes purity and minimizes negative effects on the ingredients. Only biodegradable reagents are used in ecologically-friendly processing. Naturally-processed as used herein includes, but is not limited to, distillation, condensation, extraction, steamed distillation, pressure cooking and hydrolysis.

Other examples of natural processes include: saponification which uses a strong alkali base (e.g., NaOH) to create a reaction with a fat or oil to produce soap, glycerin and water in one process; esterification and transesterification which involve reacting an alcohol and an acid or base to create safe emulsifiers, stabilizers and solubilizers that thicken, hydrate, and/or moisturize; and biofermentation which converts substances through the use of a yeast and/or a bacteria to produce nutrients and/or to purify formulations. The microorganisms typically do not survive the process.

When a component is described as being naturally-derived and naturally-processed, it is meant that the component is naturally-derived, and if any processing is performed, the processing is natural. Some naturally-derived components may not require any processing, and such components still qualify as naturally-derived and naturally processed.

Several processes dilute or change the composition of an ingredient to the point that an otherwise natural ingredient becomes “unnatural” by virtue of how it is processed. Ethoxylation, sulfonation and polymerization processes are expressly excluded from the term “naturally-processed”. For example, sulfonation uses harsh processing that involves sulfates, sulfonates and/or phosphates to create effective wetting agents for use in detergents and foaming agents. Such processes are excluded from the definition of “naturally-processed” because sulfates do not break down in the environment. Ethoxylation also uses harsh processing. A byproduct of ethoxylation is 1,4 dioxane, which is carcinogenic. In addition, such materials often degrade to form products that are toxic to aquatic life (e.g., degradation of ethoxylates to nonylphenol). One example of a common naturally-derived synthetic substitute produced by processes such as sulfonation and ethoxylation is sodium lauryl/laureth sulfate (SLS). SLS is derived from coconut oil, but is processed in such a way that does not render it an “eco-friendly, natural” ingredient as the term is used herein.

The term “GRAS” as used herein is meant to mean generally recognized as safe (GRAS) by the Food and Drug Administration (FDA) when used in accordance with the FDA's good manufacturing practices (GMP) and contain no residues of heavy metals or other contaminants in excess of tolerances set by the FDA or the EPA.

The term “biodegradable” as used herein refers to carbon containing materials that can be degraded by microbial action. The “biodegradable” materials may be tested under a recognized protocol and with tested methods of established regulatory bodies such as: EPA, EPA-TSCA, OECD, MITI or other similar or equivalent organizations in the US or internationally. Suitable non-limiting examples of test methods for biodegradation include: OECD methods in the 301-305 series. Generally, all “biodegradable” materials must meet the following limitations: (a) removal of dissolved organic carbon >70%, (b) biological oxygen demand (BOD) >60%, (c) % of BOD of theoretical oxygen demand >60%, and (d) % CO₂ evolution of theoretical >60%.

The term “eco-friendly, natural ingredient” and similar descriptors refers to an ingredient that is naturally-derived, naturally-processed, GRAS and biodegradable.

The term “cosmetic lip composition” means any lip product in a stick, tube, or jar including lip balms, tinted lip balms, squeezable lip tube products, lip gloss, lip oil, or lip products containing colorants such as lipstick. These products could be in emulsion, liquid, gel or solid form. In liquid or emulsion form, the lip balms are typically packaged into containers such as jars or wands. Gels (and sometimes liquids) are often packaged into squeeze tubes. Solid lip balms are typically molded into a bullet or stick form, or poured directly into packaging (tube or other shape).

The processing and filling method chosen will be dependent on the cosmetic lip composition, target packaging, and the desired attributes of the lip product. Solid cosmetic lip compositions are generally processed by heating the ingredients until liquid and then filled via hot pour or bullet methods. In the hot pour method, the hot liquid is poured into final packaging while hot and then cools until solidified. In the bullet method, the hot liquid is poured into molds (usually metal or silicone) where it solidifies during cooling and then placed into packaging.

In one embodiment, the lip product is a lip emulsion with two separate phases that are combined together and poured into in a stick, tube, or jar. In a second embodiment, the lip composition is hot poured into a mold upside down, the product is cooled, and then releasing agent such as sunflower oil releases the balm from the mold and then the balm is mechanically transferred into packaging. In a third embodiment, the ingredients in specific quantities are melted and mixed until uniform and then hot poured into tubes which undergo remelt cycle(s) in order to smooth the top layer.

In one embodiment, the cosmetic lip compositions of the present invention contain at least 95% naturally-derived, naturally-processed, GRAS, biodegradable ingredients. Unlike prior art formulations which include a subset of the components which may be “natural” ingredients, but which also include significant fractions of synthetic, non-eco-friendly components for purposes of emulsification, thickening, etc., the compositions of the present invention include no or essentially no synthetic, non-eco-friendly components, while exhibiting emulsification, thickening, and stability characteristics that are as good or better than those including synthetic or quasi-synthetic components.

In a second embodiment, the cosmetic lip compositions of the present invention contain industry standard ingredients that can be selected from both synthetic and natural.

### II. INTRODUCTION

Dried food powder is often used in food and nutrition products, but not commonly used in cosmetics. “Dried food powder” as used herein means a powder obtained by drying a food product. Food product examples include a whole fruit or vegetable, the juice, root, rhizome or other plant part (e.g., vanilla beans, coffee beans, etc.) The food product is turned into a powder by freeze drying or other drying method, and grinding the dried food to a powder form, however, the particle sizes of the powders may vary and included in the definition are particles that appear crystalline or may be described as crystals. Examples of dried food powders that can be used in this invention include powders obtained from the fruit juice of oranges, grapefruits, lemons, limes, raspberries, blueberries, acai berries, mangos, papayas, pineapples, strawberries, pomegranates, kiwis, vanilla beans, coffee beans and virtually any other fruit. Fillers and anti-caking agents such as silica and maltodextrin may be present in the powders to improve shelf life and prevent clumping. U.S. Pat. No. 9,243,843 which is hereby incorporated by reference describes dried food powders suitable for use in the present invention.

Cosmetic lip compositions are typically produced by heating the ingredients and filling into packaging while hot. Adding dried food powder to a cosmetic lip composition that is manufactured by hot pour is challenging because the powder is sensitive to heat and prone to settling while the composition is cooling. The thermal behavior of cosmetic lip compositions was studied and the particle size of the dried food powder was varied. A unique combination of ingredients, including a combination of oils, butters, plant based emollients (e.g., glyceryl behenate, olea europea (olive) oil unsafonifables and glyceryl rosinate), a combination of antioxidants, flavor, sweetener, dried food powder, and maltodextrin was found to provide the preferred setting temperature and deliver the desired tactile qualities. Preferred ingredients include sunflower wax and plant based emollients, such as, glyceryl behenate, olea europea (olive) oil unsafonifables and glyceryl rosinate.

Because the powder used is comprised of real food (e.g., fruit), it has the tendency to brown over time due to oxidation. The addition of one or more antioxidant compounds to the cosmetic lip composition will improve the stability. Suitable antioxidants include beta carotene, rosemary extract and tocopherol.

This invention provides a lip balm comprising of a combination of waxes, a combination of oils, behenyl olivate, shea butter, olive oil unsafonables and glyceryl rosinate, a combination of antioxidants, flavor, sweetener, dried food powder, and maltodextrin.

### III. EXEMPLARY COSMETIC LIP COMPOSITION

A. Waxes

Embodiments of cosmetic lip compositions include one or more wax components. Such waxes are generally of natural origin. Such waxes generally include a mixture of esters, cerotic acid, and hydrocarbons. Natural waxes may be extracted or derived from various natural plant sources (e.g., sunflower wax) or secreted by insects such as honey bees (e.g., beeswax). Sunflower wax is an example of a particularly preferred wax suitable for use in embodiments of the cosmetic lip compositions of the present invention.

Waxes of synthetic origin may also be used. These are generally a mix of hydrocarbons or polymeric materials that may be of mineral, petrolatum origin and produced by synthetic process. Among the synthetic waxes that may be useful include paraffin wax, polyethylene wax, microcrystalline wax, synthetic beeswax, emulsifying wax, siliconyl waxes, cersesin and ozokerite waxes. This may also include modified waxes, which are natural waxes modified by means of chemical reaction with additions or subtractions to the existing chemical structure of the molecule. These include beeswax dervatives such as PEG-6, PEG-8 beeswax, PEG-20 sorbitan beeswax, and siliconyl beeswax.

The wax may be the most prevalent component included within the composition, included in an amount greater than any other component. For example, if the cosmetic lip composition is a solid form, such as a lip balm or lip stick, the wax may be present within the composition in a range of at least 15%, preferably at least 35%, from about 15% to about 45%, preferably from about 25% to about 35%.

If the cosmetic lip composition is in a semi-solid form such as squeezable lip balm or lip gloss, the wax may be present within the composition in a range of at least 1%, preferably at least 5%, from about 1% to about 10%, preferably from about 3% to about 6%.

B. Oils

The cosmetic lip compositions according to the present invention include one or more oil component. Suitable oils include naturally derived oils such as sunflower oil, soybean oil, castor oil, olive oil, almond oil, coconut oil.

If the cosmetic lip composition is a solid form such as a lip balm stick or lip stick, the oils may be present within the composition in a range of at least 15%, preferably at least 45%, from about 15% to about 60%, preferably from about 40% to about 50%.

If the cosmetic lip composition is in a semi-solid form such as squeezable lip balm or lip gloss, the oils may be present within the composition in a range of at least 50% , preferably at least 80%, from about 50% to about 90%, preferably from about 75% to about 85%.

C. Plant Based Emollients

Plant based emollients that are highly emollient on spread and are ideal for use in oil-in-water emulsions as well as anhydrous formulations are preferred. These emollients help improve lip feel while improving stabilizing properties in the oil phase of the formulation. Plant based emollients may be present within the composition in a range of at least 0.05%, preferably at least 0.5%, from about 0.05% to about 10%, preferably from about 1% to about 5%.

One particularly preferred plant based emollient is behenyl olivate. The functional attribute for this ester complex derived from Olive oil, is that it is highly emollient on spread and ideal for use in oil-in-water emulsions as well as anhydrous formulations. This natural based ester helps improve lip feel while improving stabilizing properties in the oil phase of the formulation.

D. Fats

Fats provide emolliency, moisturization and structure to lip care products. Fats are typically plant or animal derived. Examples of fats are butters sunflower, avocado, olive and cocoa butter. Shea Butter is a particularly suitable fat for use in embodiments of the cosmetic lip compositions of the present invention.

E. Olive Oil Unsafonables/Glyceryl Rosinate

Triglyceride of natural origin and rosin glycerol ester (e.g., shea butter, olive oil, and glyceryl rosinate). This is a lanolin alternative. Animal derived lanolin or other alternatives could also be used.

F. Antioxidants

The cosmetic lip compositions according to the present invention include one or more antioxidant component. This could include vitamins such as vitamin E, F, or C, or plant extracts. Preferred antioxidants include beta carotene, rosemary extract and tocopherol. For example, the antioxidants may be present within the composition in a range of at least 0.01%, preferably at least 0.1%, from about 0.01% to about 2%, preferably from about 0.1% to about 0.5% (preferred range).

G. Dried Food Powders

Dried food powders are free-flowing powders manufactured by a freeze drying or other drying process that captures the taste of the original ingredients with minimal effect on color, flavor, or nutrients. Powders typically comprise of the dried food along with bulking agents such as maltodextrin and silicone dioxide to prevent caking. Some common examples of foods used in freeze drying are orange, raspberry, strawberry, kiwi, pineapple, ginger, apple, apricot, berry, beet, broccoli, cranberry, kale, mango, pea, peach, pear, pumpkin, grape, lemon, lime, spinach, carrot, passion fruit, pomegranate and watermelon. However, the list of foods that could be dried by freeze drying or other drying process is virtually endless. Dried food powders may be present within the composition in an amount of at least about 1%, a range from about 1% to about 20%, and preferably from about 5% to about 15%.

H. Other

Fragrances, flavors, sweeteners and maltodextrin may be added in small amounts to provide an aromatically pleasing effect. Suitable fragrances, flavors and sweeteners include those generally known to one of skill in the art. Examples include, but are not limited to, any natural or synthetic flavor or fragrance, rebaudiosides, monoammonium glycrrhizate, honey, fructose, maltodextrin and sucrose and combinations thereof.

### IV. EXAMPLES

An exemplary cosmetic lip composition containing food powder is shown below:

| Ingredient | Amount(% wt/wt) |
|---|---|
| Oil | 10-50% |
| Beeswax | 20-30% |
| Additional wax | 1-15% |
| Olea europea (olive) oil unsafonifables (and) | 0.5-5% |
| glyceryl rosinate | |
| Butyrospermum parkii (shea butter) | 0.5-5% |
| Behenyl olivate | 0.1-5% |
| Rosmarinus Officinalis (Rosmary) Extract | 0.01-2% |
| Sweetner | 0.003-0.3% |
| Dried Food Powder | 1-20% |
| Flavoring agent | 0.1-8% |
| Total: | 100.00% |

Another exemplary cosmetic lip composition containing food powder is shown below:

| Ingredient | Amount(% wt/wt) |
|---|---|
| Helianthus Annuus (Sunflower) Seed Oil | 20-30% |
| Euphorbia cerifera (candelilla) wax | 0.5-2% |
| Cocos Nucifera (Coconut) Oil | 15-25% |
| Helianthus Annuus (Sunflower) Seed Wax | 0.5-5% |
| Beeswax | 20-30% |
| Ricinus Communis (Castor) Seed Oil | 1-5% |
| Butyrospermum parkii (shea butter) (and) olea europea | 0.5-3% |
| (olive) oil unsafonifables (and) glyceryl rosinate | |
| Butyrospermum parkii (shea butter) | 1-5.0% |
| Behenyl olivate | 1-5.0% |
| Tocopherol | 0.1-0.5% |
| Rosmarinus Officinalis (Rosemary) Extract | 0.1-.05% |
| Rebaudioside A | 0.01-0.03% |
| Dried Food Powder (fruit extract, (and) silica, (and) | 5-15% |
| maltodextrin) | |
| Flavor | 1-8% |
| Total: | 100.00% |

While the present invention has been described with reference to what are presently considered to be the preferred embodiments, it is to be understood that the invention is not limited to these embodiments. To the contrary, the invention is intended to cover various modifications and equivalent arrangements included within the spirit and scope of the appended claims.

We claim:

1. A cosmetic lip composition comprising:
a wax;
an oil;
a plant based emoillient; and
a dried food powder.

2. The cosmetic lip composition recited in claim 1, wherein the wax is a natural.

3. The cosmetic lip composition recited in claim 1, wherein the cosmetic lip composition is a solid and the wax is present in a least 15% and the oil is present in a least 15%.

4. The cosmetic lip composition recited in claim 1, wherein the cosmetic lip composition is a semi-solid and the wax is present in a least 1% and the oil is present in a least 50%.

5. The cosmetic lip composition recited in claim 2, wherein the wax is sunflower wax, beeswax, candelila wax, or combinations thereof.

6. The cosmetic lip composition recited in claim 1, wherein the oil is sunflower oil, castro oil, olive oil, almond oil, coconut oil, or combinations thereof.

7. The cosmetic lip composition recited in claim 1, wherein the plant based emollient is present in at least 0.05%.

8. The cosmetic lip composition recited in claim 1, wherein the plant based emollient is benhenyl olivate.

9. The cosmetic lip composition recited in claim 1, further comprising a fat.

10. The cosmetic lip composition recited in claim 9, wherein the fat is a plant derived butter selected from the group consisting of sunflower, soybean, avocado, olive, cocoa, shea and combinations thereof.

11. The cosmetic lip composition recited in claim 1, further comprising lanolin, a lanolin alternative or combinations thereof, wherein the lanolin alternative contains a triglyceride and glyceryl rosinate.

12. The cosmetic lip composition recited in claim 11, wherein the triglyceride is shea butter, olive oil, or a combination thereof.

13. The cosmetic lip composition recited in claim 1, further comprising an antioxidant.

14. The cosmetic lip composition recited in claim 13, wherein the antioxidant is vitamin E or source thereof, vitamin F or source thereof, vitamin C or source thereof, beta carotene, rosemary extract, tocopherol, or combinations thereof.

15. The cosmetic lip composition recited in claim 1, wherein the dried food powder is derived from a fruit.

16. The cosmetic lip composition recited in claim 1, wherein the dried food powder is derived from a vegetable.

17. The cosmetic lip composition recited in claim 1, wherein the dried food powder is crystalline.
